(19) United States
(12) Patent Application Publication
(10) Pub. No.: US 2007/0158192 A1
(43) Pub. Date: Jul. 12, 2007

(54) Apparatus and method for coupling microfluidic systems with a mass spectrometer utilizing rapid voltage switching

(75) Inventors: Aaron T. Timperman, Morgantown, WV (US); James Lenke, Morgantown, WV (US); Trust Razunguzwa, Morgantown, WV (US)

Correspondence Address: PALMER & DODGE, LLP;KATHLEEN M. WILLIAMS, 111 HUNTINGTON AVENUE, BOSTON, MA, 02199, US

(73) Assignee: West Virginia University Research Corporation

(21) Appl. No.: 11/495,905

(22) Filed: Jul. 28, 2006

Related U.S. Application Data
(60) Provisional application No. 60/704,212, filed on Jul. 29, 2005.

(51) Int. Cl. H01J 49/00; C07K 1/26

(52) U.S. Cl. 204/450; 250/288

(57) ABSTRACT

The invention relates to an apparatus for coupling microfluidic systems with electrospray ionization mass spectrometry utilizing a hydrodynamic flow. The invention also relates to a method of preventing a sample in a main channel of a microfluidic device from migrating down either a first side channel or a second side channel by rapidly alternating a voltage being applied to the first side channel and the second side channel.

### RELATED APPLICATION(S)

This application claims the benefit of U.S. Provisional Application No. 60,704,212, filed on Jul. 29, 2005.

The entire teachings of the above application(s) are incorporated herein by reference.

### FIELD OF THE INVENTION

The present invention relates to microfluidic devices. More specifically, the present invention discloses an apparatus and method utilizing rapid voltage switching for delivering a sample from a main channel of a microfluidic device to a mass spectrometer without having the sample migrate down a side channel.

### BACKGROUND OF THE INVENTION

Microfluidic devices (or microchips) are devices with microchannels fabricated on glass, quartz or polymeric substances that can be engineered for sample preparation, solid phase extraction, tryptic digestions or separations. The interest in microchips stems from their ability to handle extremely small volumes with complex sample processing being achieved within the fluidic network, whose fundamental units are the zero dead volume intersections fabricated on these devices. Massive parallelism, shorter analysis time, increased separation efficiencies, higher sensitivities and reduced waste generation can be achieved with these devices.

Mass spectrometry (“MS”) is currently one of the most important analytical techniques in the analysis of biological samples, especially proteomic samples. MS provides a separation dimension according to mass/charge (“m/z”) as well as a wealth of structural information from collision induced dissociation experiments with low detection limits. Interfacing of microfluidic devices to mass spectrometry has been of great interest due to the ability of a microfluidic device to perform sample processing prior to detection by the mass spectrometer. Moreover, the flowrates in microfluidic devices (tens of nL-a few μL) are compatible with those required in nanoelectrospray MS.

Different strategies have been employed to interface microfluidic devices to mass spectrometry and early attempts involved spraying liquid directly from the microchannel exit. With this design, difficulties are experienced in controlling the size and stability of the taylor cone resulting in sample dilution, band broadening and lower sensitivities. The device also requires a high voltage to overcome the liquid surface tension and initiate electrospray.

### SUMMARY OF THE INVENTION

The present invention provides an apparatus and method for coupling microfluidic systems with electrospray ionization mass spectrometry utilizing a hydrodynamic flow. The present invention comprises a microfluidic device having a main channel which runs from an input channel to an output channel. The output channel of the present invention delivers a sample to a mass spectrometer via electrospray ionization.

The present invention provides a high voltage electrode positioned adjacent to the input channel. Further, the present invention provides a first side channel engaging the main channel at a first intersection point and a second side channel engaging the main channel at a second intersection point. An electrode is located in the first side channel and an electrode is located in the second side channel. In a preferred embodiment of the present invention, a voltage is alternatively applied (i.e., rapidly switched) to the electrode in the first side channel and the electrode in the second side channel. Rapidly alternating the voltage causes the sample to continue down the main channel and not to migrate into either the first side channel or the second side channel.

The present invention also provides a method of preventing a sample in a main channel of a microfluidic device from migrating down either a first side channel or a second side channel by rapidly alternating a voltage being applied to the first side channel and the second side channel. The method comprises delivering a sample to a microfluidic device, driving the sample towards the intersection of the first side channel and the second side channel and the main channel, rapidly switching a voltage between the first side channel and the second side channel so that the sample does not migrate down either side channel but rather continues along the main channel towards the mass spectrometer.

The apparatus and method of the present invention eliminates the need for a make-up flow solution to prevent a sample from migrating out of the main channel and into the first side channel or the second side channel. Such make-up flow solutions can dilute the sample and negatively effect results.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 shows a preferred embodiment of the present invention wherein a first side channel intersects a main channel at a first intersection point and a second side channel intersects the main channel at a second intersection point.

FIG. 2 shows a conventional application of voltage which produces band splitting.

FIG. 3A-C show a schematic representation of the present invention whereby a voltage in a first side channel and the voltage in a second side channel are rapidly switched preventing a sample to migrate down the first side channel or the second side channel.

FIG. 4 shows a preferred embodiment of the present invention whereby a switch allows for the voltage to be rapidly alternated between a first side channel and a second side channel.

FIG. 5A shows a representation of the present invention wherein a voltage is applied to the first side channel. FIG. 5B shows a representation of the present invention wherein a voltage is applied to the second side channel.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and sprit of the principles of the present invention.

### DETAILED DESCRIPTION

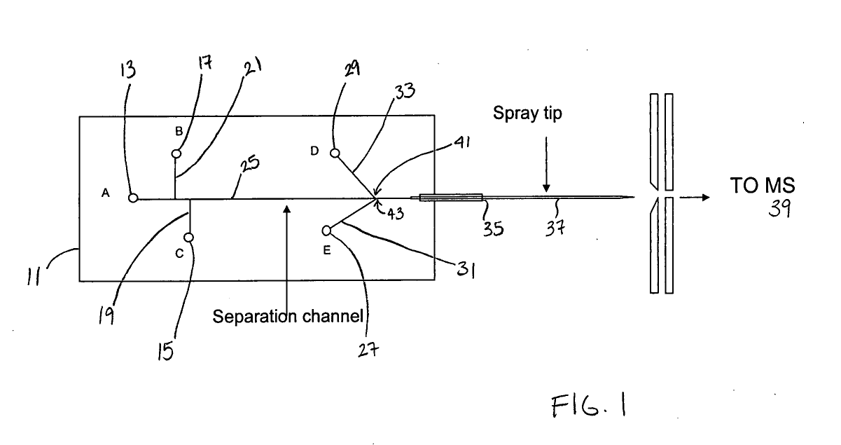

FIG. 1 shows a preferred embodiment of the present invention wherein a microfluidic device 11 is shown having a main channel 25. The main channel 25 is engaged to a first input channel 19 and a waste channel 17. The first input channel 19 is engaged to an input reservoir 15 and the waste channel 21 is engaged to a waste reservoir 17. In a preferred embodiment of the present invention, a high voltage electrode 13 is positioned adjacent to the input channel 19.

In a preferred embodiment of the present invention, the sample undergoes capillary electrophoresis which being driven down the main channel 25 towards the mass spectrometer 39. The high voltage electrode 13 provides the driving force to move the sample towards an output channel 35 and eventually into a spray tip 37 and then towards a mass spectrometer 39 via electrospray ionization (“ESI”).

In an embodiment of the present invention, the main channel 25 of the microfluidic device 11 comprises a coating. In an embodiment of the present invention, the coating is uncharged. A common problem with microfluidic devices is that the various channels of the device require a charge in order to utilize electroosmotic flow. As such, charged analytes are attracted to the charged walls of the various channels leading to sample loss. Because the present invention is not utilizing electroosmotic flow, the main channel 25 of the microfluidic device 11 may be coated with an uncharged coating to prevent such sample loss.

In a preferred embodiment of the present invention, electrodes are positioned downstream of the high voltage electrode 13 in order to better control the voltage at the spray tip 37. Such additional electrodes are critical because voltage at the ESI/MS interface is an important variable when delivering a sample to a mass spectrometer 39 for analysis (as opposed to merely relying on the voltage difference between the high voltage electrode 13 and the ESI/MS interface).

In a preferred embodiment of the present invention, a first such downstream electrode is positioned in a first side channel 31 and a second such downstream electrode is positioned in a second side channel 33. The first side channel 31 engages a first side channel reservoir 27 and the second side channel 33 engages a second side channel reservoir 29. In a preferred embodiment of the present invention, the first side channel 31 engages the main channel 25 at a first intersection point 43 and the second side channel 33 engages the main channel 25 at a second intersection point 41.

As the sample approached the first intersection point 43 and the second intersection point 41, a voltage is alternatively applied to the downstream electrode of the first side channel 31 and then to the downstream electrode of the second side channel 33. If a voltage was only applied to one of the electrodes, for example, only to the first side channel 31, the sample would be attracted to that electrode and migrate down the first side channel 31 and towards the activated electrode. Such a result is undesirable because it leads to sample loss. Rapidly switching the voltage between the first channel 31 and the second channel 33 results in the two vectors canceling each other out and the sample continuing along the main channel 25 towards the mass spectrometer 39.

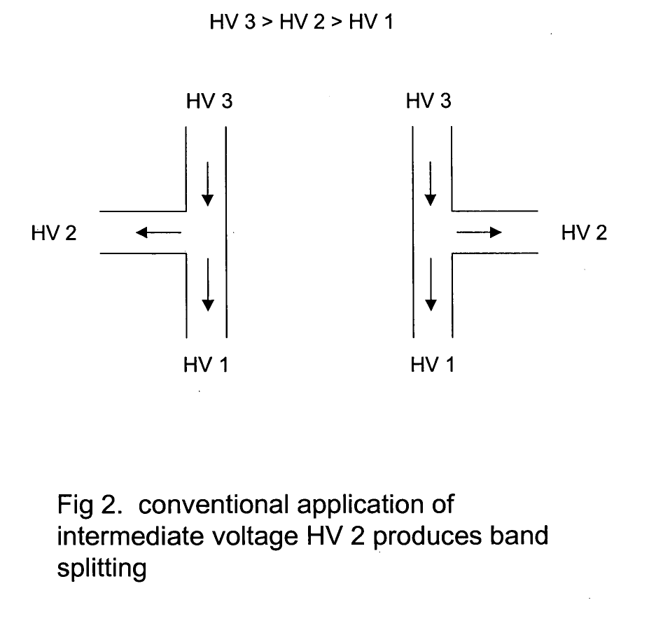

FIG. 2 shows the conventional apparatus and method of providing a single downstream electrode. FIG. 2 shows an intersection between a first side channel and a main channel. As the sample reaches the intersection point, a voltage applied to the single channel results in the sample migrating down the side channel. Sample loss down the side channel has been combated in the past by delivering a make-up solution down the side channel and into the main channel at a high rate to drive the sample towards the mass spectrometer. The downside of such an apparatus and method is that the make-up solution can dilute the sample. Further, the make-up solution may migrate towards the input channel and not towards the mass spectrometer, as desired.

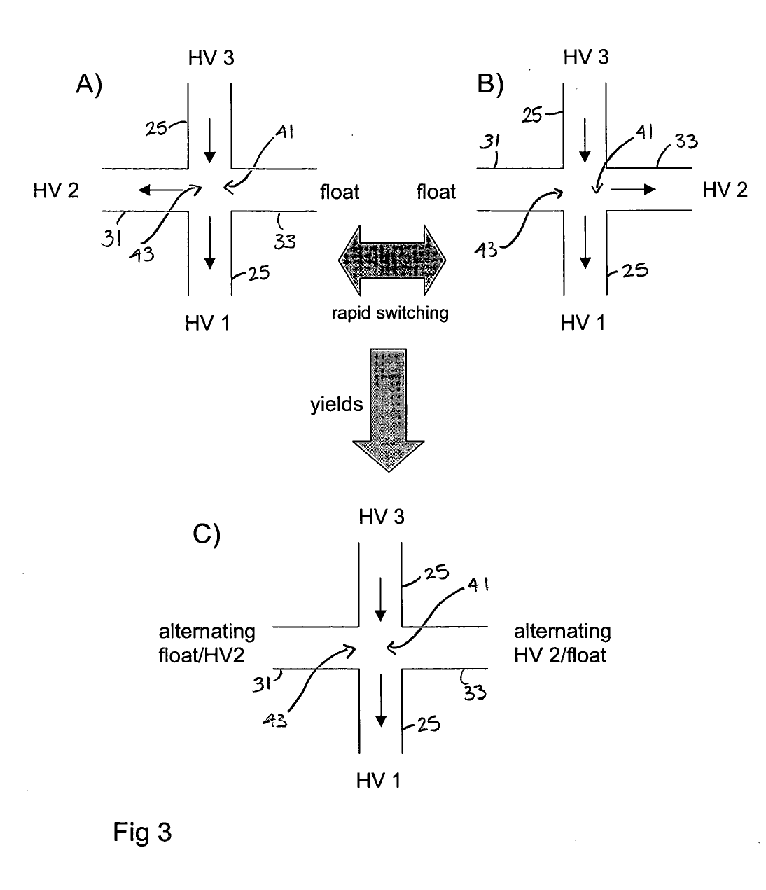

FIG. 3 shows the first side channel 31 engaging the main channel 25 at a first intersection point 43 and a second side channel 33 engaging the main channel 25 at a second intersection point 41. FIG. 3A shows a representation of a preferred embodiment of the present invention wherein a voltage is applied to a first side channel 31. FIG. 3B shows a representation of a preferred embodiment of the present invention wherein a voltage is applied to a second side channel 33 of the present invention. FIG. 3C shows the resulting flow of sample when a voltage is rapidly alternated (or switched) between the first side channel 31 and the second side channel 33. As shown in FIG. 3C, rapidly switching the voltage between the first side channel 31 and the second side channel 33 eliminates migration of the sample down either the first side channel 31 or the second side channel 33; instead, the sample continues down the main channel 25 towards the mass spectrometer 39.

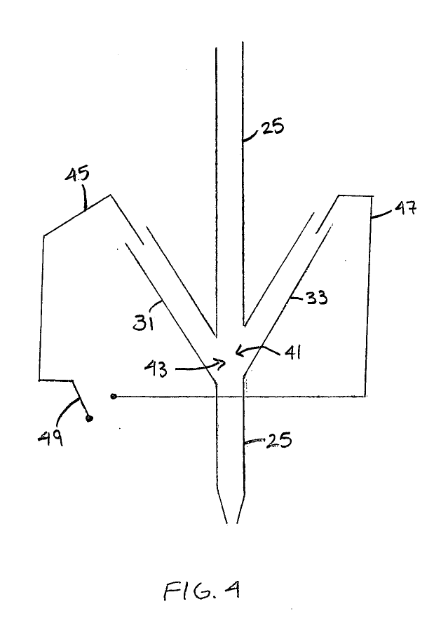

FIG. 4 show an embodiment of the present invention wherein a switch 49 is provided between the first downstream electrode 45 of the first side channel 31 and the second downstream electrode 47 of the second side channel. By rapidly opening and closing the switch 49, a voltage is alternated between the first side channel 31 and the second side channel 33 resulting in a cancellation of any migration of the sample down either side channel 31, 33. As a result, the sample continues down the main channel 25 and into the mass spectrometer 39.

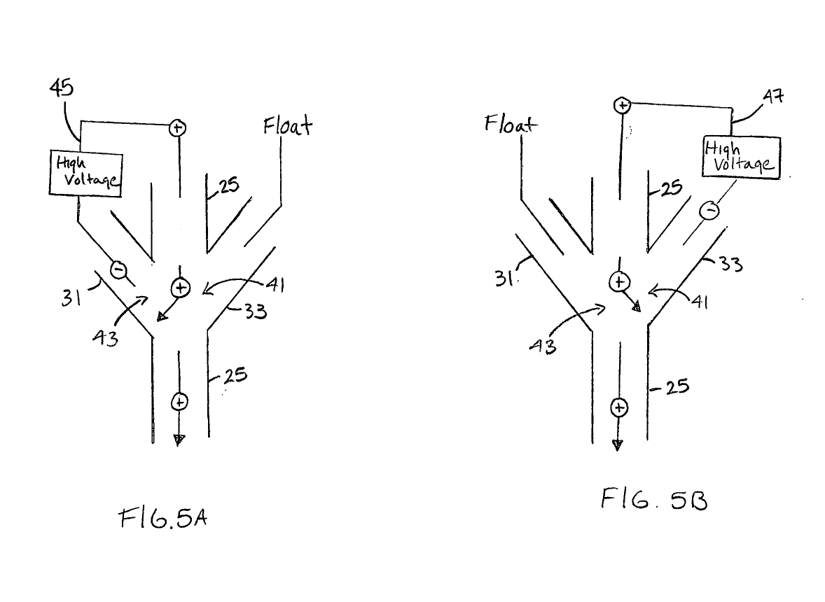

FIGS. 5A and 5B shown another representation of the present invention. FIG. 5A shows a voltage being applied to a downstream electrode 45 in communication with the first side channel 31. FIG. 5B shows a voltage being applied to a second downstream electrode 47 in communication with the second side channel 33. Rapidly alternating between the representation of FIG. 5A and the representation of FIG. 5B cancels out the migration down either channel 31, 33 and helps drive the sample towards the mass spectrometer 39.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. An apparatus for coupling microfluidic systems with electrospray ionization mass spectrometry utilizing a hydrodynamic flow, the apparatus comprising a microfluidic device having a main channel which runs from an input channel to an output channel.

2. The apparatus of claim 1, wherein said output channel delivers a sample to a mass spectrometer via electrospray ionization.

3. The apparatus of claim 1, wherein a high voltage electrode is positioned adjacent to said input channel.

4. The apparatus of claim 1, wherein a first side channel engages said main channel at a first intersection point and a second side channel engages said main channel at a second intersection point.

5. The apparatus of claim 4, wherein an electrode is located in said first side channel and an electrode is located in said second side channel.

6. The apparatus of claim 5, wherein a voltage is alternatively applied to the electrode in the first side channel and the electrode in the second side channel.

7. A method of preventing a sample in a main channel of a microfluidic device from migrating down either a first side channel or a second side channel, said method comprising:
a) delivering a sample to a microfluidic device;
b) driving the sample towards the intersection of the first side channel and the second side channel and the main channel; and
c) rapidly switching a voltage between the first side channel and the second side channel so that the sample does not migrate down either side channel and migrates along the main channel towards a mass spectrometer.
